(19) United States
(12) Patent Application Publication
(10) Pub. No.: US 2012/0022319 A1
(43) Pub. Date: Jan. 26, 2012

(54) Systems and Methods For Reducing Gastric Volume

(76) Inventor: David Muller, Boston, MA (US)

(21) Appl. No.: 12/954,456

(22) Filed: Nov. 24, 2010

Related U.S. Application Data
(60) Provisional application No. 61/264,636, filed on Nov. 25, 2009.

(51) Int. Cl. A61F 2/04

(52) U.S. Cl. 600/37

(57) ABSTRACT

Systems and methods maintain a fold, e.g., an intussusception, formed in a portion of the stomach wall. In one example, a system includes a holding device that is positioned at least partially in a stomach and receives a portion of a wall of the stomach. The holding device includes a first section connected to a second section. The holding device is operable to move the second section relative to the first section into a position in which a cavity is defined between the first section and the second section. The cavity receives the portion of the wall of the stomach. The system includes a securing device that is positionable around the second section while the portion of the stomach wall is received by the cavity. The securing device is operable to provide a force around the second section to maintain the portion of the stomach wall stably in the cavity.

### CROSS-REFERENCE TO RELATED APPLICATIONS

This application claims the benefit of U.S. Provisional Patent Application No. 61/264,636, which was filed on Nov. 25, 2009, the contents of which are entirely incorporated herein by reference.

### BACKGROUND OF THE INVENTION

1. Field of the Invention

The invention pertains to stomach procedures that treat disorders such as obesity, and more particularly, to systems and methods for reducing gastric volume.

2. Description of Related Art

A variety of treatments exist for treating obesity. One particular treatment involves reducing gastric volume by drawing a portion of the stomach wall inwardly to form a fold in the stomach wall. The fold is typically formed about the periphery of the gastroesophageal junction to define an intussusception. Intussusception generally refers to the creation of a continuous tissue fold created by telescoping one part of the stomach onto or into another part of the stomach. A portion of the stomach wall is intussuscepted to create a pouch proximal to the intussuscepted tissue. The pouch is capable of storing a volume from 0 cc up to about 100 cc. The creation of the pouch reduces the amount of food that may be ingested and may also result in a negative feedback that reduces the desire to eat, thereby aiding weight loss efforts.

Intussuscepting a portion of the stomach may be achieved by delivering an expandable device into the interior of the stomach and expanding the expandable device to define a cavity near the gastroesophageal junction. The stomach wall is drawn into the cavity, e.g., using suction, to form the intussusception and the corresponding pouch. Aspects of this procedure are described further in U.S. patent application Ser. Nos. 12/265,539 and 12/265,509, filed Nov. 5, 2008, which are continuation applications of U.S. patent application Ser. No. 11/870,096, filed Oct. 10, 2007, the contents of these applications being incorporated entirely herein by reference. In particular, these applications teach that one or more anchors are be employed to secure the intussusception and hold the pouch in place. The one or more anchors function by piercing the folded parts of the stomach wall to engage the folded parts and keep them together.

### SUMMARY OF THE INVENTION

Embodiments according to aspects of the present invention provide systems and methods for maintaining a fold, e.g., an intussusception, formed in a portion of the stomach wall. Advantageously, the embodiments provide alternatives to the use of anchors that pierce the folded parts of the stomach wall to keep the fold together, thereby avoiding the complications that may occur with piercing the stomach wall.

According to one embodiment, a system for maintaining a fold in a portion of a stomach wall includes a holding device that is positioned at least partially in a stomach and receives a portion of a wall of the stomach. The holding device includes a first section connected to a second section. The second section moves relative to the first section. The holding device is operable to move the second section relative to the first section into a position in which a cavity is defined between the first section and the second section. The cavity receives the portion of the wall of the stomach. The system also includes a securing device that is positionable around the second section while the portion of the stomach wall is received by the cavity. The securing device is operable to provide a force around the second section to maintain the portion of the stomach wall stably in the cavity between the first section and the second section. In a particular example, the holding device is deployed at or near the gastroesophageal junction, where the first section is generally tubular in shape and the second section is frustoconical in shape.

According to another embodiment, a system for maintaining a fold in a portion of a stomach wall, includes an inner device and an outer device. The outer device includes a passageway and the inner device is positionable in the passageway. A gap is defined between the inner device and the outer device. The gap receives a portion of a wall of the stomach. At least one of the outer device and the inner device applies a force radially toward the gap to maintain the portion of the stomach wall stably in the gap between the inner device and the outer device.

According to another embodiment, a method maintains a reduction in gastric volume where a fold is formed in a portion of a wall of a stomach, the fold reducing a volume of the stomach. The method includes positioning a holding device to receive a fold in a wall of a stomach. The holding device includes a first section connected to a second section. The second section is movable relative to the first section. The fold is disposed about a periphery of the first section. The method further includes moving the second section of the holding device relative to the first section into a position in which a cavity is defined between the first section and the second section. The cavity is formed to receive the fold in the wall of the stomach. Additionally, the method includes applying a securing device around the second section while the fold in the stomach wall is received by the cavity. The securing device applies a force around the second section to maintain the portion of the stomach wall stably in the cavity between the first section and the second section. In a particular example, the first section includes a tubular wall and the second section includes a frustoconical wall, and moving the second section of the holding device includes inverting the second section so that the frustoconical wall faces the tubular wall to define the cavity that receives the fold in the stomach wall. In a further example, the fold in the stomach is formed at a gastroesophageal junction of the stomach, the first section and the second section defining a passageway that is configured to lead into the stomach.

According to an additional embodiment, a method for maintaining a reduction in gastric volume includes forming a fold in a portion of a wall of a stomach, where the fold reduces a volume of the stomach. The method also includes positioning an outer device around a fold in a stomach wall, the outer device including a passageway that receives the fold in the stomach wall. The outer device applies a force radially inward to maintain the fold in the stomach wall stably. In a particular example, the method additionally includes positioning an inner device proximate to the fold in the stomach wall. The fold being disposed about a periphery of the inner device. The method positions the outer device around the inner device. The inner device is disposed in the passageway of the outer device. A gap is defined between the inner device and the outer device. The gap receives the fold in the wall of the stomach.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1A illustrates an example holding device for receiving and maintaining a fold formed in a portion of the stomach wall according to aspects of the present invention, where a movable portion of the holding device is in a first position.

FIG. 1B illustrates the holding device of FIG. 1A with a movable portion in a second position.

FIG. 1C illustrates a cross-sectional view of the holding device of FIG. 1A in its second state and engaging a portion of the stomach wall.

FIG. 2 illustrates a variation of the holding device of FIG. 1A according to aspects of the present invention.

FIG. 3 illustrates an example securing device employable with the holding device of FIG. 1A according to aspects of the present invention.

FIG. 4A illustrates an example holding system for maintaining a fold formed in a portion of the stomach wall according to aspects of the present invention.

FIG. 4B illustrates a cross-sectional view of a system employing the holding device of FIG. 2A.

FIG. 5 illustrates a cross-sectional view of another system for reducing gastric volume according to aspects of the present invention.

### DETAILED DESCRIPTION

Embodiments according to aspects of the present invention provide systems and methods for maintaining a fold, e.g., an intussusception, formed in a portion of the stomach wall. Advantageously, the embodiments provide alternatives to the use of anchors that pierce the folded parts of the stomach wall to keep the fold together, thereby avoiding the complications that may occur with piercing the stomach wall.

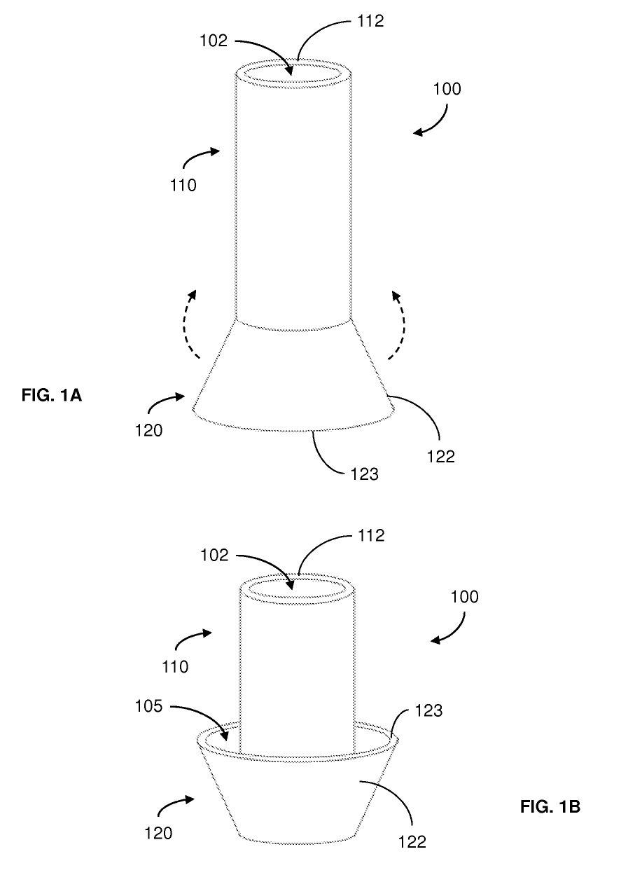

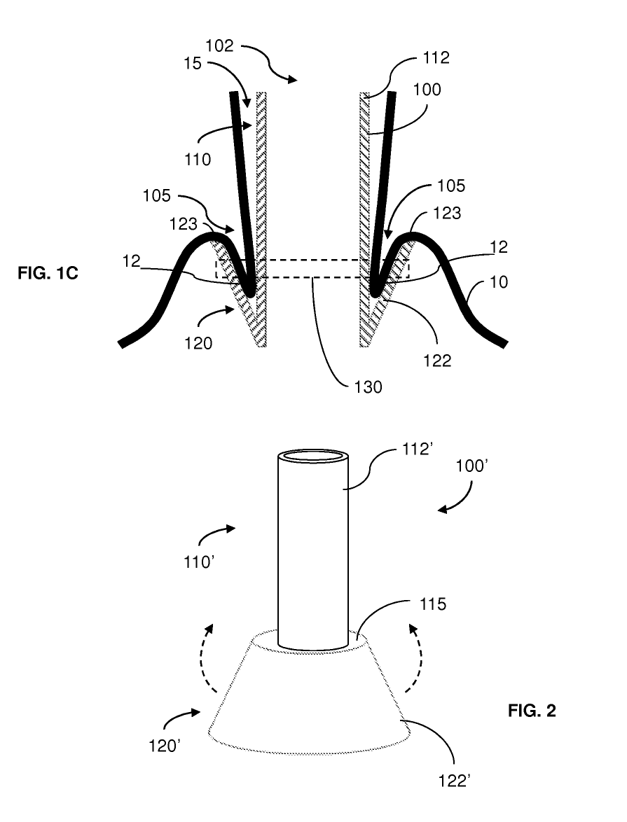

FIGS. 1A-C illustrate an example holding device 100 for receiving and maintaining a fold formed in a portion of the stomach wall. The fold, for example, may be formed in the stomach according to the teachings described previously. The fold reduces gastric volume to aid in weight loss treatments. The holding system 100 includes a first tubular section 110 and a second movable section 120. A passageway 102 passes through the tubular section 110 and the movable section 120. The tubular section 110 includes a generally tubular wall 112. The movable section 120 includes a wall 122 that defines a generally frustoconical shape. One end of the frustoconical shape is disposed at one end of the tubular section 110, while the other end of the frustoconical shape is defined by a free edge 123.

The holding device 100 employs a pliable material that allows the movable section 120 to be moved relative to the tubular section 110. FIG. 1A illustrates a first position for the movable section 120, where the movable section 120 extends away from the tubular section 110 and the free edge 123 of the movable section 120 is distal from the tubular section 110. Meanwhile, FIG. 1B illustrates a second position for the movable section 120, where the movable section 120 extends over the tubular section 110 and the free edge 123 of the movable section 120 is disposed over the tubular section 110. When the movable section 120 is in the second position, the wall 112 of the tubular section 110 and the wall 122 of the movable section 120 face each other. As such, a cavity 105 is formed between the walls 112 and 122. In general, when a sufficient amount of force is applied to the movable section 120, e.g., at the free edge 123, the movable section 120 can be flipped between the first position shown in FIG. 1A and the second position shown in FIG. 1B.

Referring to the cross-sectional view of FIG. 1C, the holding device 100 is applied to a portion of a stomach after an intussusception is formed in the stomach wall 10. In particular, the movable section 120 of the holding device 100 is in the second position. As FIG. 1C also shows, a continuous fold 12 is formed by the stomach wall 10. With the holding device 100 in place, the continuous fold 12 is received and held within the cavity 105. The tubular section 110 is disposed within the passageway 15 leading into the stomach, while the movable section 120 holds the continuous fold 12 against the tubular section 110. The tubular section 110 ensures that the passageway 15 remains open.

Although FIGS. 1A-C may show that the walls 112 and 122 may form a V-shaped cavity 105, it is understood that the walls 112 and 122 may define other shapes for the cavity 105. For example, the cavity 105 may be more contoured, e.g., U-shaped. Alternatively, the cavity 105 may have a flatter, more rectangular profile in place of the sharper angle of the V-shape. As a further alternative, the cavity 105 may have a bulbous shape in place of the sharper angle of the V-shape. In some cases, these other shapes may be more effective in accommodating the shape of the continuous fold 12 received by the cavity 105. FIG. 2 illustrates an embodiment 100′ in which the tubular section 110′ and the movable section 120′ are connected by a contoured annular section 115 that extends radially outward from the tubular section 110′, so that when the movable section 120′ is inverted, the cavity between the walls 112′ and 122′ has a wider and more contoured bottom to accommodate the fold.

As shown further in FIG. 1C, the holding device 100 is secured in place by deploying a securing device or band 130 around the walls 122 of the movable section 120. The band 130 may be an elastic material that fits in tension around the movable section 120 and imparts a force that is directed radially inward.

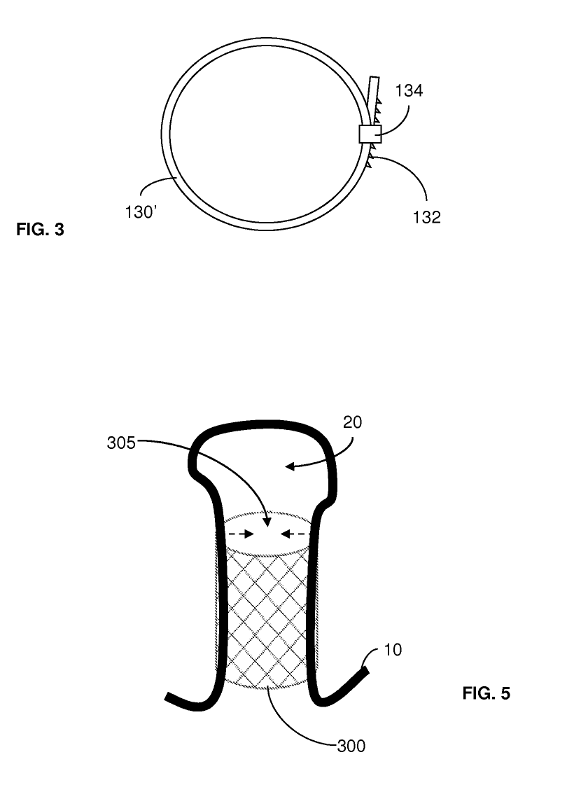

Alternatively, the band 130 may be a locking strap that is tightened mechanically around the walls 122. For example, as shown in FIG. 3, the locking strap 130′ may include a plurality of teeth 132 on one end and a locking pawl 134 on the other end. When the two ends of the locking strap are joined, the teeth 132 engage the locking pawl 134. The locking pawl 134 is configured, e.g., with oppositely angled teeth, to allow the teeth 132 to pass the locking pawl 134 easily as the locking strap is tightened around the walls 122. The locking pawl 134, however, is configured to prevent the teeth 132 from moving in the opposite direction and allowing the locking strap from loosening. Accordingly, the teeth 132 move in a ratchet-like manner so that the locking strap can provide an adjustable tension.

In addition to securing the position of the holding device 100, the compressive force ensures that the walls 112 and 122 apply sufficient frictional force to the continuous fold 12 to maintain the intussusception.

In operation, the holding device 100 may be delivered into the stomach with an endoscopic device when the intussusception is formed. The movable section 120 may be in the first position as shown in FIG. 1A when the holding device 100 enters the stomach. The movable section 120 can then be manipulated by the endoscopic device to move the movable section 120 into the second position as shown in FIG. 1B. To facilitate handling and positioning, the holding device 100 is delivered past the gastroesophageal junction. Once the movable section 120 is in the second position, the holding device 100 is retracted to engage the continuous fold 12 as illustrated in FIG. 1C. An endoscopic device also applies and manipulates the band 130 around the holding device 100 to secure it in position.

In some embodiments, the movable section 120 may be further compressed within a removable sheath when the holding device 100 is delivered into the stomach. The movable section 120 moves into the first position when the sheath is removed. Alternatively, the movable section 120 may move directly into the second position when the sheath is removed.

The holding device 100 may be subsequently removed by severing the band 130 and disengaging the holding device 100 from the continuous fold 12. Once the holding device 100 is disengaged, the movable section 120 is moved back to the first position to allow the holding device 100 to be more easily withdrawn from the stomach. In some embodiments, a sheath may be placed over the holding device 100 to further facilitate withdrawal from the stomach.

Although the movable section 120 may move between the first position shown in FIG. 1A and the second position shown in FIG. 1B, the movable section 120 provides sufficient resistance so that the moveable section 120 cannot be accidentally moved by an amount of force that occurs during normal operating conditions.

Although the examples shown in FIGS. 1A-C and 2 illustrate a first section with a generally tubular shape and a second movable section with a generally frustoconical shape, other embodiments according to aspects of the present invention are not limited to the illustrated shapes. For example, the first section may have a shape that is less cylindrical and corresponds more closely to the gastroesophageal junction. As a further example, the second section may formed from one or more sub-sections or may include one or more slits in the wall to facilitate its movement from the first position to the section position.

In general, a system for maintaining a fold in a portion of a stomach wall includes a holding device that is positioned at least partially in a stomach and receives a portion of a wall of the stomach. The holding device includes a first section connected to a second section. The second section moves relative to the first section. The holding device is operable to move the second section relative to the first section into a position in which a cavity is defined between the first section and the second section. The cavity receives the portion of the wall of the stomach. The system also includes a securing device that is positionable around the second section while the portion of the stomach wall is received by the cavity. The securing device is operable to provide a force around the second section to maintain the portion of the stomach wall stably in the cavity between the first section and the second section.

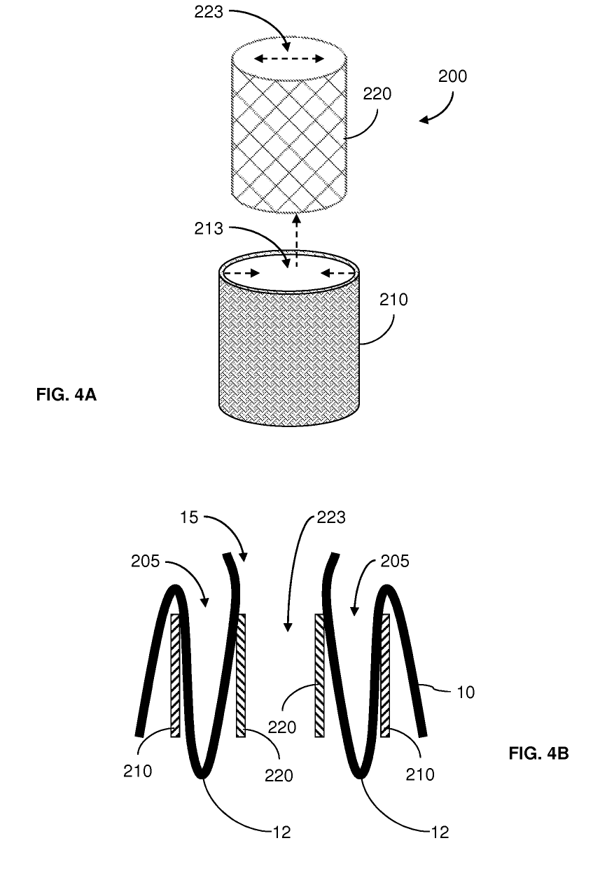

FIGS. 4A-B illustrate an example holding system 200 for maintaining a fold formed in a portion of the stomach wall. The holding device 200 includes an outer braided sheath 210 and an inner tubular device 220. The braided sheath 210, for example, may employ a biaxial braid. The braid allows the length of the braided sheath 210 to change. An increase in length is possible due to a reduction in the angle between the warp and weft threads at their crossing points. The reduction in angle, however, causes a reduction in the distance between the crossing points and a reduction in the circumference of the braided sheath 210. Therefore, axial forces that lengthen the braided sheath 210 cause the braided sheath 210 to shrink inwardly and apply an inward radial force (compression). In some aspects, the braided sheath 210 acts as a hyperbolic spring, where further reduction in axial length of the braided sheath 210 reduces the amount of radial compression.

The braided sheath 210 includes a passageway 213. When deployed, the inner tubular device 220 is positioned within the passageway 213 to define a gap 205 between the outer braided sheath 210 and the inner tubular device 220 which receives the fold in the portion of the stomach wall.

As shown in the cross-sectional view of FIG. 4B, the holding system 200 is applied to a portion of a stomach after an intussusception is formed in the stomach wall 10. In particular, the tubular device 220 is disposed within the passageway 15 into the stomach, and the braided sheath 210 is deployed so that the continuous fold 12 is held in the gap 205 between the tubular device 220 and the braided sheath 210. The tubular device 220 includes a passageway 223 and ensures that the passageway 15 into the stomach remains open. As shown in FIG. 4A, the tubular device 220 may be a stent-like device that is biased to expand radially outward to support the passageway 15 and to apply a force to hold the continuous fold 12 against the braided sheath 210. The stent-like device may be deployed in a manner similar to cardiac stents, for example. However, in some embodiments, the tubular device 220 may be a non-expanding structure that provides a passive inner support for the application of the braided sheath 210. In addition, although the embodiment of FIGS. 4A-B may illustrate particular shapes, e.g., cylindrical shapes, other embodiments are not limited to these shapes and may, for example, be shaped to accommodate the gastroesophageal junction or the fold more closely.

In operation, the holding device 200 may be delivered into the stomach with an endoscopic device when the intussusception is formed. The tubular device 220 is deployed within the passageway 15 into the stomach, near the gastroesophageal junction. The braided sheath 210 is compressed along the axial direction to shorten the axial length of the braided sheath 210. As described previously, shortening the axial length causes the circumference of the braided sheath 210 to increase. With this increased circumference, the braided sheath 210 can be positioned around the continuous fold 12. When the axial compression on the braided sheath is removed and the length of the braided sheath 210 is permitted to increase, the circumference of the braided sheath 210 decreases. Correspondingly, the braided sheath 210 applies the inward radial force to hold the continuous fold 12 against the tubular device 220. The force applied by the braided sheath 210 increases particularly if the tubular device 220 expands outwardly as a stent-like device.

The holding system 200 may be subsequently removed by cutting the braided sheath 210 and withdrawing the tubular device 220. If the tubular device 220 is a stent-like device, a sheath may be placed over the stent-like device to compress the stent-like device and further facilitate withdrawal from the stomach.

Although the tubular device 220 and the braided sheath 210 shown in FIGS. 4A-B may be separate, aspects of the tubular device 220 and the braided sheath 210 may be integrated into a single device. In particular, the tubular device 220 may extend from the passageway 15 into the stomach where the braided sheath 210 is coupled to the tubular device 220 and extends around the continuous fold 12.

In other embodiments, an elastic sleeve mat be employed in place of the braided sheath 210. Alternatively, a compressive stent-like device may be employed in place of the braided sheath 210. In this embodiment, the compressive stent-like device is applied to provide a radially inward force that holds the continuous fold 12 against the tubular device 220. In particular, after the intussusception is formed, an expanding stent-like device is deployed in the passageway 15, and a corresponding compressive stent-like device is deployed on the mucosal surface. As with the embodiments above, the stomach wall of the continuous fold 12 is squeezed together to maintain the intussusception.

As described previously, embodiments according to aspects of the present invention may employ stent-like devices that are either unidirectionally expandable or contractible. Some stent-like devices include ratchet-like teeth on the wires of the stent-like device, where the teeth allow expansion or contraction of the stent-like device but resist movement in the other direction. In some aspects, the ratcheting effect is similar to that described with respect to FIG. 3. As described previously, the stent-like device 220 may be deployed in a manner similar to cardiac stents as are known. Indeed, the expanding stent-like device 220 may be deployed by inflating a balloon within the stent-like device 220 to expand the stent-like device 220 against the walls of the passageway 15. In this case, rachet-like teeth on the wires of the stent-like device 220 would allow expansion of the stent-like device 220 but would resist compression.

In general, with reference to FIGS. 4A-B, a system for maintaining a fold in a portion of a stomach wall, includes an inner device and an outer device. The outer device includes a passageway and the inner device is positionable in the passageway. A gap defined between the inner device and the outer device receives a portion of a wall of the stomach. At least one of the outer device and the inner device applies a force radially toward the gap to maintain the portion of the stomach wall stably in the gap between the inner device and the outer device.

Similar to the holding system 200 which may employ a stent-like device, the holding device 100 illustrated in FIGS. 1A-C may also employ a stent-like device for the tubular section 110. In this embodiment, a band 130 may be optional, as the stent-like device may provide a sufficient force against the passageway to keep the holding device 100 securely in place.

Although embodiments according to aspects of the present invention may provide systems and methods for maintaining an intussusception, other embodiments according to aspects of the present invention provides systems and methods for reducing gastric volume without an intussusception. Referring to FIG. 5, a pouch 20 is formed by sucking in any portion of the stomach wall 10 and maintaining the pouch 20 by applying a compressive stent-like device 300 while the pouch is disposed in the passageway 305 defined by the device 300. An inner supporting structure, similar to the tubular structure 220, is not required because there is no passageway to keep open. Moreover, the gastric volume can be further reduced by employing more than one compressive stent-like device 300 within the stomach. In alternative embodiments, the device 300 may be replaced by the other compressive structures, e.g., the elastic sleeve, band, braided sheath, etc., described previously.

While the present invention has been described in connection with a number of exemplary embodiments, and implementations, the present inventions are not so limited, but rather cover various modifications, dimensions, shapes, and equivalent arrangements. Other implementations of the invention will be apparent to those skilled in the art from consideration of the specification and practice of the invention disclosed herein. Various aspects and/or components of the described embodiments may be used singly or in any combination. It is intended that the specification and examples be considered as exemplary only. In general, the embodiments may employ various tools and devices, e.g., laparoscopic end effectors, suitable for deploying and removing the devices. Moreover, the embodiments form the devices from materials that are suitable for their intended function within the gastric environment, and that are preferably removable.

1. A system for maintaining a fold in a portion of a stomach wall, comprising:
a holding device configured to be positioned at least partially in a stomach and receive a portion of a wall of the stomach, the holding device including a first section connected to a second section, the second section being movable relative to the first section, wherein the holding device is operable to move the second section relative to the first section into a position in which a cavity is defined between the first section and the second section, the cavity being configured to receive the portion of the wall of the stomach; and
a securing device that is positionable around the second section while the portion of the stomach wall is received by the cavity, the securing device being operable to provide a force around the second section to maintain the portion of the stomach wall stably in the cavity between the first section and the second section.

2. The system of claim 1, wherein the first section includes a tubular wall and the second section includes a frustoconical wall, the holding device being operable to invert the second section so that the frustoconical wall faces the tubular wall to define the cavity that receives the portion of the stomach wall.

3. The system of claim 1, wherein the holding device is configured to be positioned at a gastroesophageal junction of the stomach, the first section and the second section defining a passageway that is configured to lead into the stomach.

4. The system of claim 1, wherein the securing device is an elastic band that is positionable in tension around the holding device.

5. The system of claim 1, wherein the securing device is a locking strap that is tightened mechanically around the holding device.

6. The system of claim 5, wherein the locking strap includes teeth that are ratcheted to tighten mechanically around the holding device.

7. A system for maintaining a fold in a portion of a stomach wall, comprising:
an inner device; and
an outer device including a passageway, the inner device being positionable in the passageway, a gap being defined between the inner device and the outer device, the gap being configured to receive a portion of a wall of the stomach, at least one of the outer device and the inner device applying a force radially toward the gap to maintain the portion of the stomach wall stably in the gap between the inner device and the outer device.

8. The system of claim 7, wherein the outer device includes a braided sheath defined by a biaxial braid that applies the radial force toward the inner device when the braided sheath is lengthened axially.

9. The system of claim 7, wherein the outer device includes an elastic sleeve.

10. The system of claim 7, wherein the outer device is mechanically adjustable to apply the force radially toward the inner device.

11. The system of claim 10, wherein the outer device includes teeth that are ratcheted to mechanically adjust the outer device to apply the force radially toward the inner device.

12. The system of claim 7, wherein the inner device is mechanically adjustable to apply the other force radially toward the outer device.

13. The system of claim 12, wherein the inner device includes teeth that are ratcheted to mechanically adjust the inner device to apply the force radially toward the outer device.

14. The system of claim 7, wherein the inner device includes a substantially tubular wall, the inner device defining another passageway that is configured to pass through a gastroesophageal junction of the stomach.

15-30. (canceled)
